(19) United States
(12) Patent Application Publication
(10) Pub. No.: US 2008/0113413 A1
(43) Pub. Date: May 15, 2008

(54) Expression of Foreign Cellulose Synthase Genes in Photosynthetic Prokaryotes (Cyanobacteria)

(75) Inventors: David R. Nobles, Austin, TX (US); R. Malcolm Brown, Austin, TX (US)

Correspondence Address: CHALKER FLORES, LLP, 2711 LBJ FRWY, Suite 1036, DALLAS, TX, 75234, US

(73) Assignee: BOARD OF REGENTS, THE UNIVERSITY OF TEXAS SYSTEM, Austin, TX (US)

(21) Appl. No.: 11/866,852

(22) Filed: Oct. 3, 2007

Related U.S. Application Data
(60) Provisional application No. 60/849,363, filed on Oct. 4, 2006.

(51) Int. Cl. C12P 19/00; C12N 1/20; C12M 1/00

(52) U.S. Cl. 435/72; 435/252.3; 435/292.1

(57) ABSTRACT

The present invention includes compositions and methods for making and using cyanobacteria that include a portion of an exogenous cellulose operon sufficient to express cellulose. The compositions and methods of the present invention may be used as a new global crop for the manufacture of cellulose, CO₂ fixation, for the production of alternative sources of conventional cellulose as well as a biofuel and precursors thereof.

### CROSS-REFERENCE TO RELATED APPLICATIONS

This application claims priority to U.S. Provisional Patent Application Ser. No. 60/849,363, filed Oct. 4, 2006, the entire contents of which are incorporated herein by reference.

### TECHNICAL FIELD OF THE INVENTION

The present invention relates in general to the field of exogenous gene expression, and more particularly, to the expression of exogenous cellulose synthase genes in cyanobacteria.

### BACKGROUND OF THE INVENTION

Without limiting the scope of the invention, its background is described in connection with cellulose production.

Cellulose biosynthesis has a significant impact on the environment and human economy. The photosynthetic conversion of CO₂ to biomass is primarily accomplished through the creation of the cellulosic cell walls of plants and algae (Lynd et al., 2002). With approximately 10¹¹ tons of cellulose created and destroyed annually (Hess et al., 1928), this process ameliorates the adverse effects of increased production of greenhouse gasses by acting as a sink for CO₂ (Brown, 2004). Although cellulose is synthesized by bacteria, protists, and many algae; the vast majority of commercial cellulose is harvested from plants.

Timber and cotton are the primary sources of raw cellulose for a number of diverse applications including textiles, paper, construction materials, and cardboard, as well as cellulose derived products such as rayon, cellophane, coatings, laminates, and optical films. Wood pulp from timber is the most important source of cellulose for paper and cardboard. However, extensive processing is necessary to separate cellulose from other cell wall constituents (Klemm et al. 2005; Brown, 2004). Both the chemicals utilized to extract cellulose from associated lignin and hemicelluloses from wood pulp and the waste products generated by this process pose serious environmental risks and disposal problems (Bajpai, 2004). Additionally, the cultivation of other cellulose sources, such as cotton, entails the extensive use of large tracts of arable land, fertilizers and pesticides (both of which require petroleum for their manufacture), and dwindling fresh water supplies for irrigation.

### SUMMARY OF THE INVENTION

More particularly, the present invention includes compositions, methods, systems and kits for the production of microbial cellulose using cyanobacterium that include a portion of an exogenous cellulose operon sufficient to express bacterial cellulose. Examples of cyanobacteria for use with the present invention include those that are photosynthetic, nitrogen-fixing, capable of growing in brine, facultative heterotrophs, chemoautotrophic, and combinations thereof. One specific example of a cyanobacterium for use with the present invention is the photosynthetic cyanobacterium Synechococcus sp. While any bacterial cellulose operon may be used alone or in combination with plant cellulose genes, one specific operon for use with the present invention is the portion of the cellulose operon sufficient to express bacterial cellulose that includes the acsAB genes from the cellulose synthase operon stably integrated into the chromosome, e.g., a cellulose operon with an exogenous promoter such as P_{lac}-acsABΔC. Other examples of cellulose operon include an acsABCD operon under control of a PrbcL promoter from Synechococcus leopoliensis, and/or that of the acsABCD operon from Acetobacter strain NQ5.

A wide variety of cellulose operon and promoter system may be used with the present invention, e.g., the cellulose operon acsABCD from NQ5 under the control of an PrbcL promoter from Synechococcus leopoliensis, a portion of the cellulose operon sufficient to express bacterial cellulose that includes the acsAB genes from the cellulose synthase operon of Acetobacter sp. or a portion of the cellulose operon sufficient to express bacterial cellulose comprises the acsAB genes from the cellulose synthase operon of the gram negative bacterium Acetobacter xylinum. In yet another embodiment, the portion of the cellulose operon sufficient to express bacterial cellulose may include the acsAB genes from the cellulose synthase operon of the gram negative bacterium Acetobacter xylinum. In another embodiment, the portion of the cellulose operon sufficient to express bacterial cellulose may include the acsAB genes from the cellulose synthase operon to produce a multi-ribbon cellulose or the acsAB genes from the cellulose synthase operon of the Acetobacter multiribbon strain NQ 5. It has been found that using the present invention it is possible to manufacture cellulose with a lower crystallinity than wild-type bacterial cellulose, amorphous cellulose, crystalline native cellulose I, regenerated cellulose II, nematic ordered cellulose, a glucan chain association, chitin, curdlan, β-1,3 glucan, chitosan, cellulose acetate and combinations thereof.

In one embodiment of the present invention, the cellulose genes are from mosses (including Physcomitriella), algae, ferns, vascular plants, tunicates, and combinations thereof. In yet another non-exclusive embodiment, the cellulose genes are selected from gymnosperms, angiosperms, cotton, switchgrass and combinations thereof. The skilled artisan will recognize that it is possible to combine portions of the operons of bacterial, algal, with fungal and plant cellulose genes to maximize production and/or change the characteristics of the cellulose.

The present invention also includes a vector for expression of a portion of the cellulose operon sufficient to express bacterial cellulose operon that includes a microbial cellulose operon, e.g., the acsAB gene operon, under the control of a promoter that expresses the genes in the operon in cyanobacteria. The skilled artisan will recognize that the vector may combine portions of the operons of bacterial, algal, fungal and plant cellulose operons to maximize production and/or change the characteristics of the cellulose and may be transfer and/or expression vector.

The present invention also includes a method of producing cellulose by expressing in a photosynthetic cyanobacterium a portion of the cellulose operon sufficient to express bacterial cellulose and isolating the cellulose produced by a photosynthetic cyanobacterium. The cyanobacterium may be a photosynthetic cyanobacterium that includes a portion of the cellulose operon sufficient to express bacterial cellulose that includes the acsAB genes from the cellulose synthase operon stably integrated into the chromosome. The cyanobacterium could be Synechococcus sp. as an example. One advantage of the present invention is that it permits the large scale manufacture of cellulose using cyanobacteria adapted for growth in ponds or enclosed photobioreactors. For example, the present invention may include growth and harvesting of cellulose grown in vast areas of brine.

The compositions and methods of the present invention also include the use of the cyanobacteria-produced cellulose, which has a lower crystallinity than wild-type bacterial cellulose and allows for easier degradation to glucose for use in subsequent fermentation to ethanol. One distinct advantage of the present invention is that it permits very large scale production of cellulose in areas that would otherwise not be available for cellulose production (e.g., areas with little or no rainfall) while at the same time producing cellulose with less toxic byproducts such as chemicals required to remove lignin and other non-cellulosic components. The cellulose of the present invention has a lower crystallinity than wild-type bacterial cellulose and the lower crystallinity cellulose is degraded with less energy into glucose than wild-type cellulose and is further converted into ethanol.

One example of the present invention is a Synechococcus cyanobacterium that has one been modified to include one or more genes from the acsAB cellulose synthase operon from a bacterium under the control of a promoter such that the cyanobacterium expresses bacterial cellulose. The cyanobacteria can be used in a system for the manufacture of bacterial cellulose that includes growing an exogenous cellulose expressing cyanobacterium in ponds and harvesting from the ponds the cyanobacterium.

The system for the manufacture of bacterial cellulose may further include growing an exogenous cellulose expressing cyanobacterium adapted for growth in a hypersaline environment, such that the cyanobacterium does not grow in fresh water or the salinity of sea water. The growth of the cyanobacterium in a hypersaline environment may be used as way to limit the potential for unplanned growth of the cyanobacteria outside controlled areas. In one example, the cellulose expressing cyanobacteria of the present invention may be grown in brine ponds obtained from subterranean formation, such a gas and oil fields. Examples of cyanobacteria for use with the system include those that are photosynthetic, nitrogen-fixing, capable of growing in brine, facultative heterotrophs, chemoautotrophic, and combinations thereof. As with the previous embodiments of the present invention, the cellulose genes may even obtained from mosses such as Physcomitriella, algae, ferns, vascular plants, tunicates, gymnosperms, angiosperms, cotton, switchgrass and combinations thereof. The skilled artisan will recognize that it is possible to combine portions of the operons of bacterial with algal, fungal and plant cellulose genes to maximize production and/or change the characteristics of the cellulose.

### BRIEF DESCRIPTION OF THE DRAWINGS

For a more complete understanding of the features and advantages of the present invention, reference is now made to the detailed description of the invention along with the accompanying figures and in which:

FIG. 1 shows a colony PCR screen for S. leopoliensis::Plac-acsABΔC. Lane 1 DNA Ladder, Lane 2 wild-type colony, Lanes 3-6 Plac-acsABΔC transgenic colonies, Lane 9 NQ5 DNA, and Lane 10 pSAB2 plasmid DNA.

FIG. 2 is a Western blot with total proteins using anti-AcsB antibody. Lane 1-A. xylinum, Lane 2-wild-type S. leopoliensis, Lanes 3 and 4-S. leopoliensis::Plac-acsABΔC mutants. Bands of significant molecular weights are labeled.

FIG. 3 shows epifluorescence micrographs of S. leopoliensis wild-type, S. leopoliensis::P_{lac}-acsABΔC, and S. leopoliensis::P_{rbcL}-acsABCD strains labeled with Tinopal. (A): Tinopal labeling of wild-type strain displaying fluorescence consistent with fluorophore penetration of dead cells. (B): S. leopoliensis::P_{lac}-acsABΔC transgenic strain depicting labeling of extracellular material with Tinopal. Cell viability is evidenced by the autofluorescence of chlorophyll. Note the elongated cells. (C): S. leopoliensis::P_{rbcL}-acsABCD transgenic strain depicting labeling of extracellular material with Tinopal. Cell viability is evidenced by the autofluorescence of chlorophyll.

FIG. 4 shows transmission electron microscopy (TEM) images of S. leopoliensis negative stained and labeled with CBHI-gold. (A): Wild-type cell displaying amorphous extracellular material. (B): Wild-type cell showing modest gold labeling at the periphery of the extracellular material shown in (A). (C): S. leopoliensis::P_{lac}-acsABΔC with CBHI-gold labeled extracellular material. (D): Higher magnification view of the labeling nearest the cell in (C) showing labeling of fibrillar material resembling crystalline cellulose.

FIG. 5 shows a colony Screen for S. leopoliensis::P_{rbcL}-acsABCD. Lanes 1-4 transgenic colonies, Lane 5 wild-type colony, and Lane 6 DNA ladder.

FIG. 6 is a transmission electron microscopy (TEM) micrographs depicting the extracellular matrices enclosing the cells of S. leopoliensis::P_{rbcL}-acsABCD. (A): A low magnification micrograph demonstrating the poles of two cells connected by matrix material is shown here. (B): The poles of two cells connected by matrix material are shown here at a higher magnification. Note the labeling of matrix material with CBHI-gold.

FIG. 7 shows the extracellular material produced by S. leopoliensis::P_{rbcL}-acsABCD labeled with CBHI-gold. (A) and (B): CBHI-gold labeling of fine aggregated material is shown in these micrographs; (C) and (D): Fibrillar material resembling crystalline cellulose is shown here labeled with CBHI-gold.

FIG. 8 is a comparison of extracellular material observed with negative staining and CBHI-gold labeling in wild-type and S. leopoliensis::P_{rbcL}-acsABCD transgenic strains. (A): Extracellular material secreted by wild-type cells is seen in this low magnification electron micrograph. (B): A higher resolution image shows the amorphous nature of the wild-type extracellular material. Note the homogeneity, as well as lack of substructure and CBHI-gold labeling. (C) and (D): Low magnification images depicting extracellular material of S. leopoliensis::P_{rbcL}-acsABCD (corresponds to the fine aggregated material seen in FIG. 7).

FIG. 9 shows a diagram of a production plant that may be used to produce, isolate and process the saccharides produced using the present invention.

FIG. 10 shows photobioreactor design for in situ harvest of cyanobacterial saccharides.

FIG. 11 is a side view of a photobioreactor complex design for in situ harvest of cyanobacterial saccharides.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term, “cellulose” and “cellulose substrate” include not only bacterial cellulose, but also native cellulose from any source such trees, cotton, any vascular plant (angiosperms and gymnosperms), any non-vascular plant such as algae, mosses, liverworts, any animal that synthesizes cellulose (such as tunicates or sea squirts), any prokaryotic organism (such as cyanobacteria, purple bacteria, archaebacteria, etc. A complete list and classification is available from the present inventors at: <128.83.195.51/cen/library/tree/cel.htm>. As the inventors' list shows, the cellulose may be from an organism that has one or more cellulose synthase genes present. Furthermore, cellulose also includes any derivatized form of cellulose such as cellulose nitrate, acetate, carboxymethylcellulose, etc. Cellulose also includes any form of native crystalline cellulose, which includes not only the native crystalline form (called cellulose I, in its alpha and beta sub allomorphs, all ratios, whether pure alpha or pure beta). Cellulose for use with the present invention also includes all processed crystalline celluloses, which deviates from the native form of cellulose I, such as cellulose II (which is a precipitated crystalline allomorph that is thermodynamically more stable than cellulose I). Cellulose includes all variations of molecular weights ranging from the lowest (oligosaccharides, 2-50 glucan monomers in a B-1,4 linkage to form a glucan chain), low molecular weight celluloses with a degree of polymerization (dp), which is the number of glucose molecules in the chain, of 50 to several hundred, on up to the highest dp celluloses known (e.g., 15,000 from some Acetobacter strains, to 25,000 from some algae). The present invention may also use all variations of non crystalline cellulose, including but not limited to, nematic ordered cellulose (NOC).

As used herein, the terms “continuous method” or “continuous feed method” refer to a fermentation method that includes continuous nutrient feed, substrate feed, cell production in the bioreactor, cell removal (or purge) from the bioreactor, and product removal. Such continuous feeds, removals or cell production may occur in the same or in different streams. A continuous process results in the achievement of a steady state within the bioreactor. As used herein, the term “steady state” refers to a system and process in which all of these measurable variables (i.e., feed rates, substrate and nutrient concentrations maintained in the bioreactor, cell concentration in the bioreactor and cell removal from the bioreactor, product removal from the bioreactor, as well as conditional variables such as temperatures and pressures) are relatively constant over time.

As used herein, the terms “photobioreactor,” “photoreactor,” or “cyanobioreactor,” include a fermentation device in the form of ponds, trenches, pools, grids, dishes or other vessels whether natural or man-made suitable for inoculating the cyanobacteria of the present invention and providing to one or more of the following: sunlight, artificial light, salt, water, CO₂, H₂O, growth media, stirring and/or pumps, gravity or force fed movement of the growth media. The product of the photobioreactor will be referred to herein as the “photobiomass”. The “photobiomass” includes the cyanobacteria, secreted materials and mass formed into, e.g., cellulose whether intra or extracellular.

As used herein, the terms “bioreactor,” “reactor,” or “fermentation bioreactor,” include a fermentation device that includes of one or more vessels and/or towers or piping arrangement, which includes the Continuous Stirred Tank Reactor (CSTR), Immobilized Cell Reactor (ICR), Trickle Bed Reactor (TBR), Bubble Column, Gas lift Fermenter, Static Mixer, or other device suitable for gas-liquid contact. A fermentation bioreactor for use with the present invention includes a growth reactor which feeds the fermentation broth to a second fermentation bioreactor, in which most products, e.g., alkanols or furans are produced. In some cases, the gaseous byproduct of fermentation, e.g., CO₂, can be pumped back into the photobioreactor to recycle the gas and promote the formation of saccharides by photosynthesis. To the extent that heat is generated during the process of recovering the products of the fermentation, etc., the heat can also be used to promote cyanobacterial cell growth and production of saccharides.

As used herein, the term “nutrient medium” refers to conventional cyanobacterial growth media that includes sufficient vitamins, minerals and carbon sources to permit growth and/or photosynthesis of the cellulose producing cyanobacteria of the present invention. Components of a variety of nutrient media suitable to the use of this invention are known and reported in e.g., Cyanobacteria, Volume 167: (Methods in Enzymology) (Hardcover), by John N. Abelson Melvin I. Simon and Alexander N. Glazer (Editors), Academic Press, New York (1988).

As used herein, the term “cell concentration” refers to the dry weight of cyanobacteria per liter of sample. Cell concentration is measured directly or by calibration to a correlation with optical density.

As used herein, the term “saccharide production” refers to the amount of mono-, di-, oligo or polysaccharides produced by the modified-cyanobacteria of the present invention that produce saccharides by fixing carbon such as CO₂ by photosynthesis into the saccharides. One distinct advantage of the present invention is that the cyanobacteria do not produce lignin along with the production of the cellulose and other saccharides that can be used a feed-stock for fermentation and other bioreactors that convert the saccharides into, e.g., ethanol or other synfuels.

In operation, the present invention may use any of a variety of known fermentation process steps, compositions and methods for converting the saccharides into useful products, e.g., lignin-free cellulose, alkanols, furans and the like. One non-limiting example of a process for producing ethanol by fermentation is a process that permits the simultaneous saccharification and fermentation step by placing the saccharide source at a temperature of above 34° C. in the presence of a glucoamylase and a thermo-tolerant yeast.

In this example, the following main process stages may be included saccharification (if necessary), fermentation and distillation. One particular advantage of the present invention is that it eliminates a variety of processing steps, including, milling, bulk-fiber separations, recovery or treatments for the control or elimination of lignin, water removal, distillation and burning of unwanted byproducts. Any of the process steps of alcohol production may be performed batchwise, as part of a continuous flow process or combinations thereof.

Saccharification. To produce mono- and di-saccharides from the lignin-free cellulose of the present invention the cellulose can be metabolized by cellulases that provide the yeast with simple saccharides. This “saccharification” step include the chemical or enzymatic hydrolysis of long-chain oligo and polysaccharides by enzymes such as cellulase, glucoamylases, alpha-glucosidase, alkaline, acid and/or thermophilic alpha-amylases and if necessary phytases.

Depending on the length of the polysaccharides, enzymatic activity, amount of enzyme and the conditions for saccharification, this step may last up to 72 hours. Depending on the feedstock, the skilled artisan will recognize that saccharification and fermentation may be combined in a simultaneous saccharification and fermentation step.

Fermentation. Any of a wide-variety of known microorganism may be used for the fermentation, fungal or bacterial. For example, yeast may be added to the feedstock and the fermentation is ongoing until the desired amount of ethanol is produced; this may, e.g., be for 24-96 hours, such as 35-60 hours. The temperature and pH during fermentation is at a temperature and pH suitable for the microorganism in question, such as, e.g., in the range about 32-38° C., e.g. about 34° C., above 34° C., at least 34.5° C., or even at least 35° C., and at a pH in the range of, e.g., about pH 3-6, or even about pH 4-5. The skilled artisan will recognize that certain buffers may be added to the fermentation reaction to control the pH and that the pH will vary over time.

The use of a feed stock that includes monosaccharides and disaccharides, in addition to the use of thermostable acid alpha-amylases or a thermostable maltogenic acid alpha-amylases and invertases in the saccharification step may make it possible to improve the fermentation step. When using a feedstock that includes large amounts of saccharides such as glucose and sucrose, for the production of ethanol it may be possible to reduce or eliminate the need for the addition of glucoamylases in the fermentation step or prior to the fermentation step.

Distillation. To complete the manufacture of final products from the saccharides made by the cyanobacterial fixation of CO₂ of the present invention, the invention may also include recovering the alcohol (e.g., ethanol). In this step, the alcohol may be separated from the fermented material and purified with a purity of up to e.g. about 96 vol. % ethanol can be obtained by the process of the invention.

Several specific enzymes and methods may be used to improve the recovery of energy containing molecules from the present invention. The enzymes improve the saccharification and fermentation steps by selecting their most efficient activity as part of the processing of the products of the saccharide producing modified cyanobacteria of the present invention.

In one example, a thermo tolerant cellulase may be introduced into the reactor to convert cellulose produced by the cyanobacteria of the present invention into monosaccharides, which will mostly be glucose. Examples of thermophilic cellulases are known in the art as taught in, e.g., U.S. Patent Application No 20030104522 filed by Ding, et al. that teach a thermal tolerant cellulase from Acidothermus cellulolyticus. Yet another example is taught by U.S. Patent Application No. 20020102699, filed by Wicher, et al., which teaches variant thermostable cellulases, nucleic acids encoding the variants and methods for producing the variants obtained from Rhodothermus marinus. The relevant portions of each are incorporated herein by reference.

Acid cellulase may be obtained commercially from manufacturers such as Ideal Chemical Supply Company, Memphis Term., USA; Americos Industries Inc., Gujarat, India; or Rakuto Kasei House, Yokneam, Israel, Novozyme, Denmark. For example, the acid cellulase may be provided in dry, liquid or high-active abrasive form, as is commonly used in the denim acid washing industry using techniques known to the skilled artisan. For example, Americos Cellscos 450 AP is a highly concentrated acid cellulase enzyme produced using a genetically modified strains of Trichoderma reesii. Typically, the acid cellulases function in a pH range or 4.5-5.5.

Microorganisms used for fermentation. One example of a microorganism for use with the present invention is a thermo-tolerant yeast, e.g., a yeast that when fermenting at 35° C. maintains at least 90% of the ethanol yields and 90% of the ethanol productivity during the first 70 hours of fermentation, as compared to when fermenting at 32° C. under otherwise similar conditions. One example of a thermotolerant yeast is a yeast that is capable of producing at least 15% V/V alcohol from a corn mash comprising 34.5% (w/v) solids at 35° C. One such thermo-tolerant yeast is Red Star®/Lesaffre Ethanol Red (commercially available from Red Star®/Lesaffre, USA, Product No. 42138). The ethanol obtained using any known method for fermenting saccharides (mono, di-, oligo or poly) may be used as, e.g., fuel ethanol, drinking ethanol, potable neutral spirits, industrial ethanol or even fuel additives.

Examples of ethanol fermentation from sugars are well-known in the art as taught by, e.g., U.S. Pat. No. 4,224,410 to Pemberton, et al. for a method for ethanol fermentation in which fermentation of glucose and simultaneous-saccharification fermentation of cellulose using cellulose and a yeast are improved by utilization of the yeast Candida brassicae, ATCC 32196; U.S. Pat. No. 4,310,629 to Muller, et al., that teaches a continuous fermentation process for producing ethanol in which continuous fermentation of sugar to ethanol in a series of fermentation vessels featuring yeast recycle which is independent of the conditions of fermentation occurring in each vessel is taught; U.S. Pat. No. 4,560,659 to Asturias for ethanol production from fermentation of sugar cane that uses a process for fermentation of sucrose wherein sucrose is extracted from sugar cane, and subjected to stoichiometric conversion into ethanol by yeast; and U.S. Pat. No. 4,840,902 to Lawford for a continuous process for ethanol production by bacterial fermentation using pH control in which a continuous process for the production of ethanol by fermentation of an organism of the genus Zymomonas is provided. The method of Lawford is carried out by cultivating the organism under substantially steady state, anaerobic conditions and under conditions in which ethanol production is substantially uncoupled from cell growth by controlling pH in the fermentation medium between a pH of about 3.8 and a pH less than 4.5; and K A Jacques, T P Lyons & D R Kelsall (Eds) (2003), The Alcohol Textbook; 4^{TH} Edition, Nottingham Press; 2003. The relevant portions of each of which are incorporated herein by reference.

One of ordinary skill in the art would recognize that the quantity of yeast to be contacted with the photobiomass will depend on the quantity of the photobiomass, the secreted portions of the photobiomass and the rate of fermentation desired. The yeasts used are typically brewers' yeasts. Examples of yeast capable of fermenting the photobiomass include, but are not limited to, Saccharomyces cerevisiae and Saccharomyces uvarum. Besides yeast, genetically altered bacteria know to those of skill in the art to be useful for fermentation can also be used. The fermenting of the photobiomass is conducted under standard fermenting conditions.

Separating of the ethanol from the fermentation can be achieved by any known method (e.g. distillation). The separation can be performed on either or both the liquid and solid portions of the fermentation solution (e.g., distilling the solid and liquid portions), or the separation can just be performed on the liquid portion of the fermentation solution (e.g., the solid portion is removed prior to distillation). Ethanol isolation can be performed by a batch or continuous process. The separated ethanol, which will generally not be fuel-grade, can be concentrated to fuel grade (e.g., at least 95% ethanol by volume) via additional distillation or other methods known to those of skill in the art (e.g., a second distillation).

The level of ethanol present in the fermentation solution can negatively affect the yeast/bacteria. For example, if 17% by volume or more ethanol is present, then it will likely begin causing the yeast/bacteria to die. As such, ethanol can be separated from the fermentation solution as the ethanol levels (e.g., 12, 13, 14, 15, 16, to 17% by volume (ethanol to water)) that may kill the yeast or bacteria are reached. Ethanol levels can be determined using methods known to those of ordinary skill in the art.

The fermentation reaction can be run multiple times on the photobiomass or portions thereof. For example, once the level of ethanol in the initial fermentation reactor reaches 12-17% by volume, the entire liquid portion of the fermentation solution can be separated from the biomass to isolate the ethanol (e.g., distillation). The “once-fermented” photobiomass can then be contacted with water, additional enzymes and yeast/bacteria for additional fermentations, until the yield of ethanol is undesirably low. Factors that the skilled artisan will use to determine the number of fermentations include: the amount of photobiomass remaining in the vessel; the amount of carbohydrate remaining, the type of yeast or bacteria, the temperature, pH, salt concentration of the media and overall ethanol yield. If any carbohydrates remain, then the remaining photobiomass is removed from the vessel.

Generally, it is desirable to isolate or harvest the yeast/bacteria from the fermentation reaction for recycling. The method of harvesting will depend upon the type of yeast/bacteria. If the yeast/bacteria are top-fermenting, they can be skimmed off the fermentation solution. If the yeast/bacteria are bottom-fermenting, they can be removed from the bottom of the tank.

Often, a by-product of fermentation is carbon dioxide, which is readily recycled into the photobioreactor for fixation into additional saccharides. During the fermentation process, it is expected that about one-half of the decomposed starch will be discharged as carbon dioxide. This carbon dioxide can be collected by methods known to those of skill in the art (e.g., a floating roof type gas holder) and is supplied back into the photobioreactor pool or pools. In colder climates, the heat that may accompany the carbon dioxide will help in the growth of the cyanobacterial pools.

One advantage of the present invention is that it provides a novel CO₂ fixation method for the recycling of environmental greenhouse gases. The present invention provides a source of substrate for cellulose production from carbon dioxide that is fixed into sugar by photosynthesis, thereby removing a major barrier limiting large global scale production of cellulose. If successful on a large scale, this new global cellulose crop will sequester CO₂ from the air, thus reducing the potential greenhouse gas responsible for global warming. Another benefit of the present invention is that forests and cotton crops, the present sources for cellulose, may not be needed in the future, thus freeing the land to allow regeneration of forests and use of cropland for other needs.

Microbial cellulose stands as a promising possible alternative to traditional plant sources. The α proteobacterium Acetobacter xylinum (synonym Gluconacetobacter xylinum [Yamada et al., 1997]) is the most prolific of the cellulose producing microbes. The NQ5 strain (Brown and Lin, 1990) is capable of converting 50% of glucose supplied in the medium into an extracellular cellulosic pellicle (R. Malcolm Brown, Jr., personal communication). Although it possesses the same molecular formula as cellulose derived from plant sources, microbial cellulose has a number of distinctive properties that make it attractive for diverse applications. The cellulose synthesized by A. xylinum is “spun” into the growth medium as highly crystalline ribbons with exceptional purity, free from the contaminating polysaccharides and lignin found in most plant cell walls (Brown et al., 1976). The resulting membrane or pellicle is composed of cellulose with a high degree of polymerization (2000-8000) and crystallinity (60-90%) (Klemm et al., 2005). Contaminating cells are easily removed, and relatively little processing is required to prepare membranes for use. In its never-dried state, the membrane displays exceptional strength and is highly absorbent, holding hundreds of times its weight in water (White and Brown, 1989). A. xylinum cellulose is therefore, well suited as a reinforcing agent for paper and diverse specialty products (Shah and Brown, 2005; Czaja et al., 2006; Tabuchi et al., 2005; Helenius et al., 2006).

The acsAB genes from the cellulose synthase operon of or the gram negative bacterium, Acetobacter xylinum (=Gluconacetobacter xylinus) under control of a lac promoter have been integrated into the chromosome of a photosynthetic cyanobacterium, Synechococcus leopoliensis UTCC 100. The presence of the genes in the chromosome has been confirmed by PCR. Preliminary data from Western analysis, light microscopy, and growth characteristics suggests functional expression of these genes in Synechococcus. Cyanobacteria expressing exogenous cellulose synthase genes will be used for the efficient and inexpensive production of bacterial cellulose. The present invention can be used in the biosynthesis of cyanobacterial cellulose with a crystallinity and a degree of polymerization (DP) similar to that of Acetobacter cellulose for use in specialized cellulose applications.

Despite it superior quality, the use of microbial cellulose as a primary constituent for large scale use in common applications such as the production of construction materials, paper, or cardboard has not been economically feasible. The root cause for the expense of microbial cellulose production is the heterotrophic nature of A. xylinum. Bacterial cultures must be supplied with glucose, sucrose, fructose, glycerol, or other carbon sources produced by the cultivation of plants. Increased distance from the primary energy source is inherently less efficient and inevitably leads to increased cost of production when compared with phototrophic sources. Therefore, while the unique properties of A. xylinum cellulose make it indispensable for a number of value added products, it is not well suited for the more general applications that constitute the vast majority of cellulose utilization (Brown, 2004; White and Brown, 1989), e.g., to replace the use of forests for the production of paper and to provide substrates for the production of biofuels based on ethanol using photosynthesis as the source of energy for CO₂ fixation. As such, the present invention provides compositions and methods for the manufacture of a new global crop that may be used for energy production and removal of the greenhouse gas CO₂ using an environmentally acceptable natural process that requires little or no energy input for manufacture.

Currently, bacterial cellulose is produced by A. xylinum, a heterotrophic a proteobacterium. The fact that the precursor of cellulose, namely glucose, needs to be supplied, presents a bottleneck in large scale production of microbial cellulose. Present technology would suggest using sugarcane extracts, sucrose, beet sugar, etc., as sources. If the rate of cellulose biosynthesis in cyanobacteria is increased via the expression of exogenous cellulose synthase genes, then the potential for an economical global cellulose crop is possible. Cellulose synthase genes have been stably integrated into the chromosome by recombination but also could be expressed on replicating plasmids.

Unlike A. xylinum, cyanobacteria require no fixed carbon source for growth. Additionally, many cyanobacteria are capable of nitrogen fixation, which would eliminate the need for fertilizers necessary for cellulose crops like cotton. Furthermore, many cyanobacteria are halophilic, that is, they can grow in a the range of brackish to hypersaline environments. This feature, in combination with N-fixation, will allow non-arable, sun-drenched areas of the planet to provide the extensive surface areas for the growth and harvest of cellulose made using the compositions and methods of the present invention on a global scale.

Cyanobacterial cellulose can be used in diverse applications where a combination of products is simultaneously made from photosynthesis. Value added products may include pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. These products may be the result of natural cyanobacterial metabolic processes or be induced through genetic engineering. The present invention permits large scale production of cellulose, proteins and other products that may be grown and harvested. In fact, wide application of the cells themselves for glucose and cellulose is encompassed by the present invention. The cellulose producing cyanobacteria of the present invention may be utilized for energy recycling and recovery, that is, the cells may be dried and burned to power downstream processes in a manner similar to the use of bagasse in the sugar cane industries.

The ideal cellulose producing organism would synthesize cellulose of a quality and in the quantities observed in A. xylinum, have a photoautotrophic lifestyle, and possess the ability to grow with a minimum use of natural resources in environments unsuitable for agriculture. Cyanobacteria are capable of using low photon flux densities for carbon fixation, withstanding hypersaline environments, tolerating desiccation, and surviving high levels of UV irradiation (Vincent, 2000; Wynn-Williams, 2000). Additionally, many species are diazotrophic (Castenholz and Waterbury, 1989). This combination of exceptional adaptive characteristics has made mass cultivation of cyanobacteria attractive for production of nutritional biomass, fatty acids, bioactive compounds, and polysaccharides (Cogne et al., 2005; Moreno et al., 2003; Kim et al., 2005). Although no species of cyanobacteria are known to synthesize cellulose in large quantities, the development of a number of systems for engineering of cyanobacterial chromosomes may offer a means to a new global crop of cellulose produced by cyanobacteria.

Toward this end, genes that include the cellulose synthase operon of A. xylinum NQ5 were integrated into the chromosome of the unicellular cyanobacterium, Synechococcus leopoliensis UTCC 100 (synonym Synechococcus elongatus PCC 7942). Alternatively, a cyanobacterium for use with the present invention may be a salt-water variety that is diazotrophic. S. elongatus has served as a model organism for molecular studies of photosynthesis and circadian rhythms, and has been successfully utilized for transgenic expression (Rixin and Golden, 1993; Nair et al., 2000; Deng and Coleman, 1999; Asada et al., 2000). S. elongatus has a rapid growth rate, readily recombines DNA into its chromosome by transformation or conjugation, can act as a host for replicating plasmids, and its physiology, genetics, and biochemistry are well characterized (Golden et al., 1987; Thiel, 1995; Deng and Coleman, 1999). Additionally, a project to sequence the genome of this organism is underway (genomejgi-psf.org/finished_microbes/synel/synel.home.html). These characteristics facilitate the transfer and expression of exogenous genes and manipulation of native regulatory components.

### EXAMPLE 1

### Synechococcus leopoliensis::P_{lac}-acsABΔC

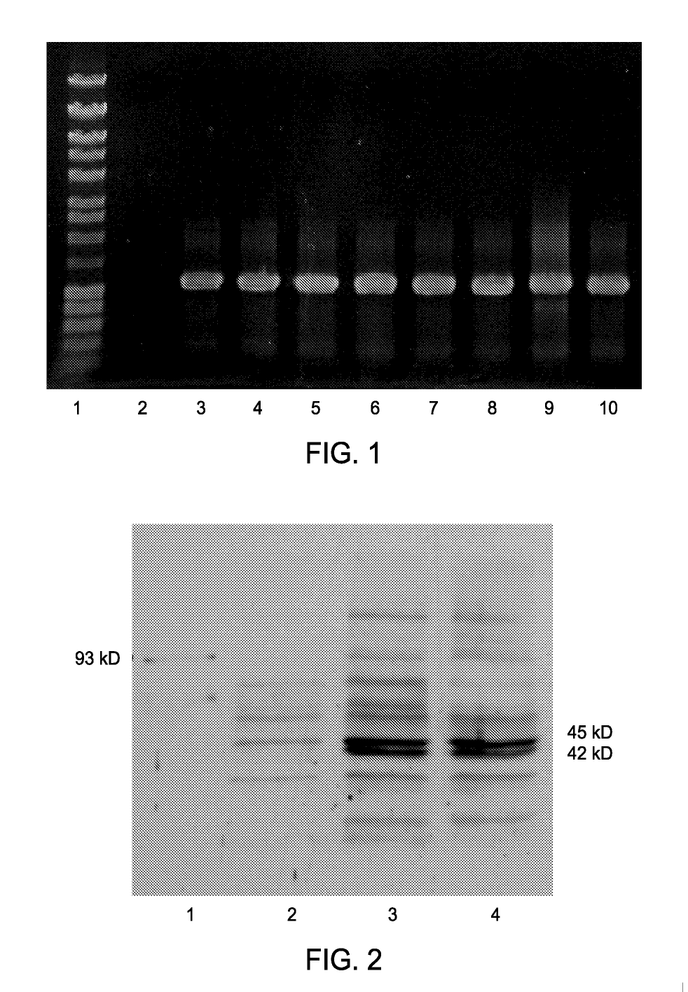

Exconjugate colonies determined to be free from E. coli contamination were used for screening of genomic integration and expression analysis. Integration of the A. xylinum NQ5 acsABΔC sequence into the neutral site (genomic region discovered in S. elongatus PCC 7942 which can be interrupted without a change in cell phenotype) of the genome of S. leopoliensis is clearly shown by a positive PCR screen (FIG. 1). The acsABΔC fragment is under the transcriptional control of the lac promoter from E. coli which results in low level constitutive expression of AcsAB. The results of a Western blot with the anti-93 kD protein (AcsB) antibody (FIG. 2) demonstrates the presence of a faint 93 kD band in both the AY201 lanes and S. leopoliensis::P_{lac}-acsABΔC lanes with no band of this size present in the UTCC100 wild type lane was observed. However, there are multiple bands present in both wild type and mutant lanes. The S. leopoliensis::P_{lac}-acsABΔC lanes show two prominent bands of 45 and 42 kD. The 45 kD band is also present in the wild type. Since searches against the genomic database of S. elongatus PCC 7942 (genomejgi-psf.org/finished_microbes/synel/synel.home.html) yield no sequences with significant similarity to AcsB, this likely represents nonspecific binding of the antibody. However, the 42 kD band is present only in the mutant lanes and may indicate products of protein degradation or processing. These data provide firm evidence that the AcsAB proteins of A. xylinum are successfully translated in the S. leopoliensis host cell.

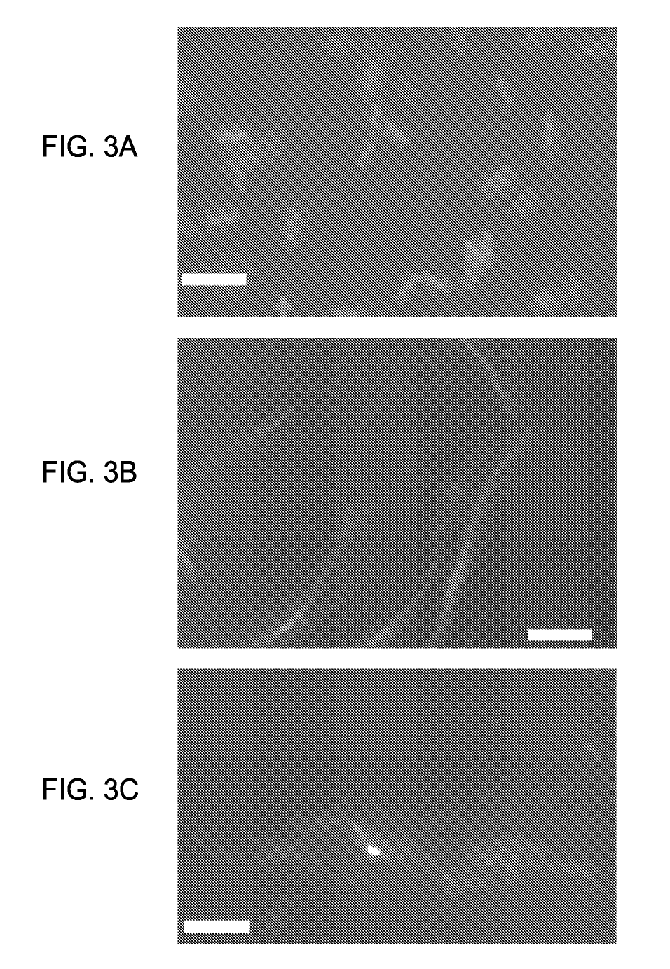

Tinopal labeling of wild-type S. leopoliensis did not indicate the presence of extracellular polysaccharides. There was limited labeling of whole cells. This often occurs when dead cells become permeable to the fluorophore and is generally not indicative of the presence of polysaccharides (FIG. 3). S. leopoliensis::P_{lac}-acsABΔC however, demonstrated labeling consistent with the secretion of an extracellular polysaccharide. The secretion of the product appears to take place laterally at sites on the long axis, as well as at the polar regions of the cells. The viability of these cells was easily monitored by the autofluorescence of chlorophyll, thus eliminating the possibility of fluorophore infiltration due to the permeability of dead cells. This phenomenon is observed in only a small population of cells, indicating that production of the positively labeled material is not synchronous in the culture. The mutant cells are often highly elongated as compared to the wild-type, a characteristic sometimes observed in S. leopoliensis as a response to stress (hence its alternative moniker S. elongatus). It is possible that since AcsA is an integral membrane protein, even low level constitutive expression causes a stress response in these cells.

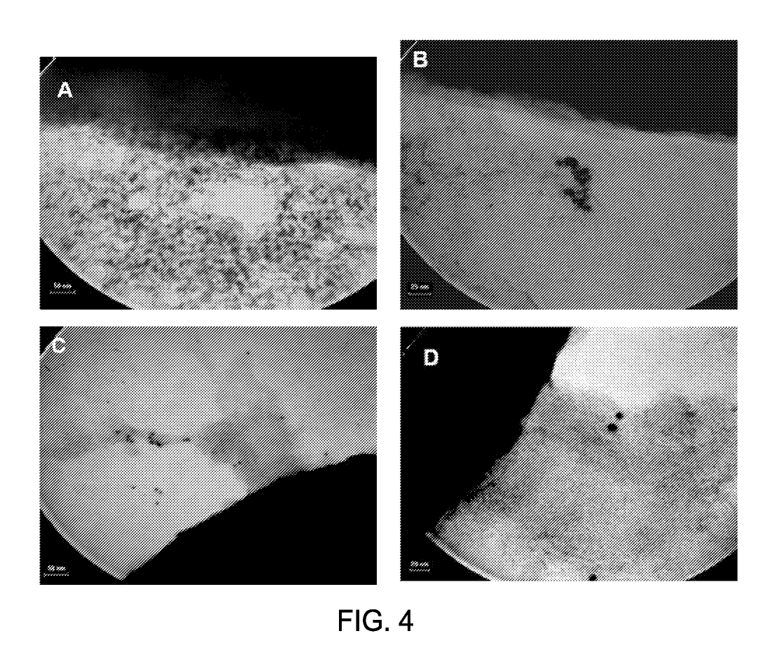

TEM examination of CBHI-gold labeled cells revealed the presence of noncrystalline material with modest labeling in wild-type cells. S. leopoliensis::P_{lac}-acsABΔC displayed material that was positively labeled. The large amount of unorganized material with chain-like substructure is reminiscent of glucan chain aggregates. Regions exist within this material with fibrillar morphology resembling crystalline cellulose (FIG. 4). The presence of even trace amounts of cellulose I would necessitate proximal orientation and at least rudimentary organization of the sites of secretion. It is also possible that some of the aggregation could be antiparallel in which case this material, if sufficiently crystalline, could be cellulose II. The cellulose of the present invention is more amenable to enzymatic degradation to glucose and thus facilitates the production of ethanolic biofuels.

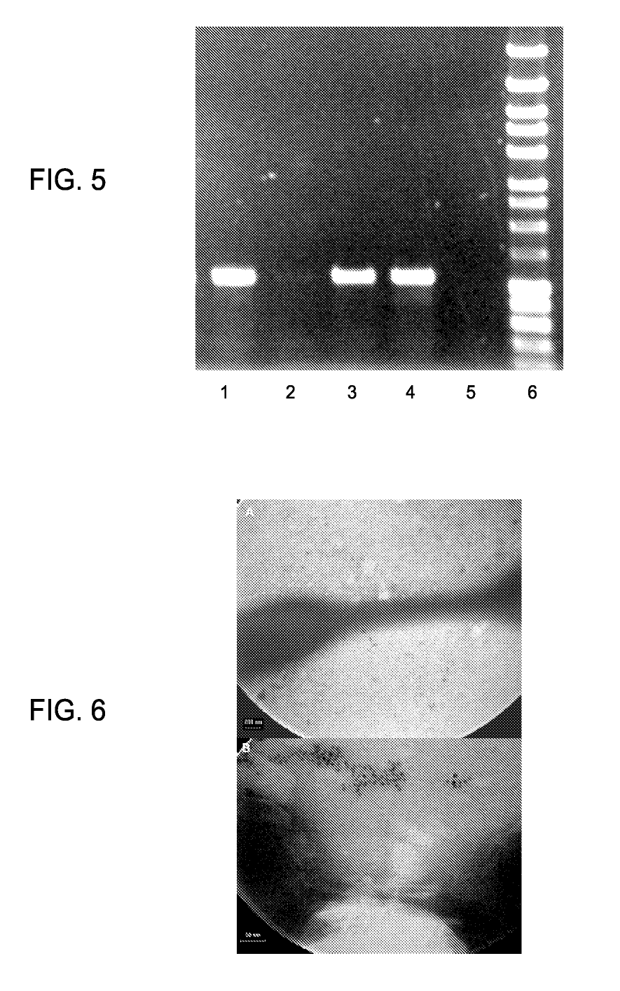

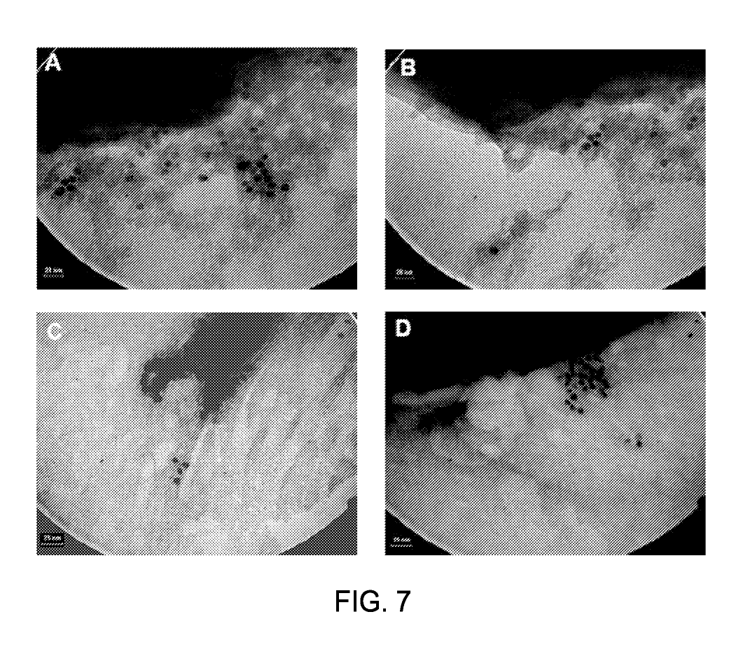

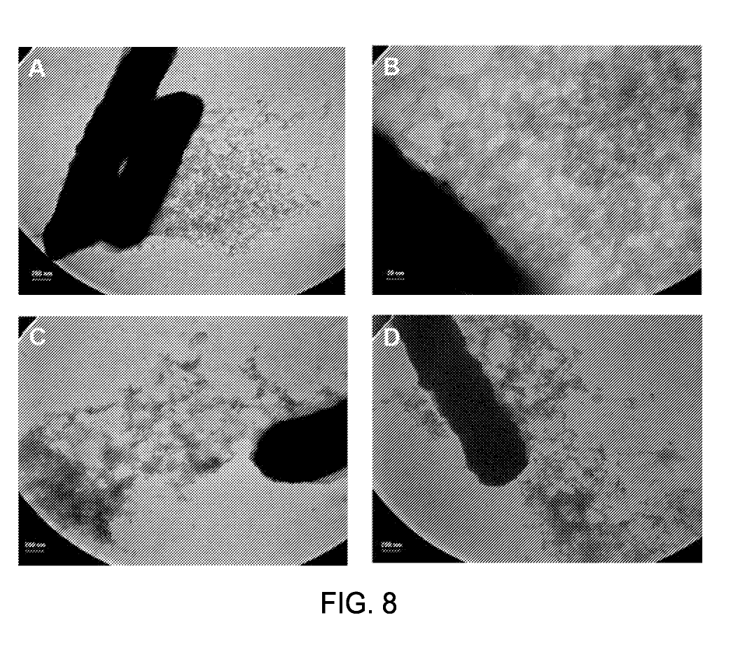

Synechococcus leopoliensis::P_{rbcL}-acsABCD. The integration of the acsABCD operon into the neutral site of S. leopoliensis was verified in the same manner as with S. leopoliensis::P_{lac}-acsABΔC (FIG. 5). Examination of Tinopal labeled wild-type S. leopoliensis collected from agar plates showed a small amount of fluorescent material. However, fluorescence did not appear to emanate from secreted material. Rather, the labeling of whole cells displayed here is indicative of dead cells. Labeling of S. leopoliensis::P_{rbcL}-acsABCD grown on plates demonstrated extracellular material similar to that observed in S. leopoliensis::P_{lac}-acsABΔC. FIG. 3 shows several cells aligned and attached to a positively labeled product. Fluorescence in mutant samples does not seem to emanate from cell permeability to Tinopal, but rather from an extracellular layer apparently acting to cause cell aggregation. The apparent encasement of cells in an extracellular matrix was confirmed with TEM examination, where cells often appeared to be connected by an extracellular matrix (FIG. 6). The matrix material consisted primarily of a fine network resembling glucan chains and small fibrils consistent with chain aggregation or low level crystallinity (FIGS. 7 and 8) similar to the material observed in S. leopoliensis::P_{lac}-acsABΔC. Labeling was light, although consistent in areas with fibrillar material. Wild-type cells were comparatively much less aggregated, but also showed the presence of extracellular material. This material appeared homogeneous, was not fibrillar, and lacked any discernable substructure; however, there was light labeling with CBHI-gold.

The sequence of the cellulose synthase operon of A. xylinum NQ5 was first elucidated twelve years ago (Saxena et al., 1994). Given this long time frame, there is surprisingly little knowledge of the molecular mechanisms of microbial cellulose biosynthesis. A positive allosteric activator of cellulose biosynthesis, cyclic diguanylic acid (c-di-GMP) has been identified, as have the enzymes responsible for regulating its concentration—diguanylate cyclase and its cognate phosphodiesterase (Ross et al., 1986; Ross et al., 1987; Tal et al., 1998; Weinhouse et al., 1997). Although AcsB is widely believed to regulate cellulose synthesis by binding c-di-GMP, of the four proteins encoded by this operon, only AcsA (the catalytic subunit) has an experimentally proven function (Lin and Brown, 1989; Weinhouse et al., 1997; Tal et al., 1998; Romling et al., 2005). While AcsC, AcsD, and an endoglucanase seem to be necessary for normal synthesis of cellulose I microfibrils, their precise function in this process remains a mystery (Saxena, 1994). This, in brief, represents the sum total of current knowledge of the enzymes involved in regulation, product catalysis, and crystallization of cellulose in A. xylinum.

The characterization of cellulose biosynthesis in other bacteria gives some insight into the minimum requirements for cellulose production. AcsA and acsB are conserved in all known proteobacterial operons encoding proteins for cellulose biosynthesis (Romling, 2002). Although these enzymes are necessary for cellulose synthesis in the Enterobacteriaceae, they are not sufficient to this end. It is known that the cellulose synthase operon is constitutively transcribed in E. coli, yet cellulose is only produced under specific conditions (Zogaj et al., 2001). Control of this process is tightly controlled by regulatory proteins that contain the conserved GGDEF and EAL motifs associated with diguanylate cyclases and phosphodiesterases (Tal et al. 1998; Nikolskaya et al., 1993).

The cellulose produced by E. coli and Salmonella spp. appears as a noncrystalline aggregation of glucan chains in close association with hydrophobic fimbriae constituting the extracellular matrix of the rdar multicellular morphotype (unpublished observations, this lab). Therefore, in addition to regulatory and catalytic proteins, other yet unidentified components necessary for the production of a crystalline cellulose product must exist. It is likely that the highly regular alignment of pores that make up the terminal complex of the cells of A. xylinum is critical for crystallization (Saxena et al., 1994; Zaar, 1979). It is important to note that unlike the products observed in E. coli and Salmonella spp. which encase the cells in a cocoon-like structure (unpublished observations, this laboratory), contact of an A. xylinum cell to its product is generally limited to the unilateral secretion sites oriented parallel to the long axis (Brown et al., 1976). The fact that E. coli and Salmonella spp. cells are embedded in their extracellular matrix connotes a randomly dispersed rather than a discrete, orderly, and aligned orientation of secretion sites on the cell surface. It is important to note that even in acsD mutants of A. xylinum which produce crystalline cellulose II in addition to cellulose I, a linearly arranged row of cellulose synthesizing pores is still observed (Saxena et al., 1994). It is possible that close association of glucan chains upon secretion is necessary for the regular formation of any crystallite.

The creation of mutant strains of S. leopoliensis by integration of P_{lac}-acsABΔC and P_{rbcL}-acsABCD into the NSII site of the genome represents the first attempts at functional the cellulose synthesizing machinery from A. xylinum NQ5 in a heterologous system. Examination of these mutants demonstrates distinct phenotypic differences from the wild-type. Both the S. leopoliensis::P_{lac}-acsABΔC and S. leopoliensis::P_{rbcL}-acsABCD strains showed Tinopal labeling consistent with the production of an extracellular polysaccharide. The presence of similar material was not observed in wild-type cells. Chain aggregates, representing the majority of the extracellular material observed in both strains, were revealed in TEM examinations (FIGS. 4, 6, and 7). The dimensions and morphology of these were quite similar to the glucan chain aggregates produced by E. coli and Salmonella spp. Additionally, small amounts of fibrillar material resembling crystalline cellulose were interspersed within randomly oriented chain aggregates.

The present invention includes the functional expression of genes from the cellulose synthase operon of A. xylinum NQ5 in S. leopoliensis UTCC 100. Culture Conditions. Cultures of Synechococcus leopoliensis UTCC 100 were maintained in 50 ml or 500 ml liquid cultures in BG11 medium on a rotary shaker (Allen, 1968). Solid media was prepared as BG11 with 1% or 1.5% agar (Difco) with the addition of 1 mM Sodium Thiosulfate (Golden, 1988). Cultures were grown with 12 hour light/dark cycles at 28° C. When necessary, chloramphenicol was used for selection at a concentration of 7.5 ug/ml. E. coli strains were grown in Luria-Bertani medium at 37° C. on a rotary shaker or on 2% agar plates. For selection of resistance markers, antibiotics were used at the following concentrations: ampicillin (50 ug/ml), chloramphenicol (25 ug/ml), and tetracycline (12.5 ug/ml). A. xylinum (AY201) and A. xylinum ATCC 53582 were grown in SH medium as previously described (Shram and Hestrin, 1954). A summary of the strains and plasmids used in this study is shown in Table 1.

TABLE 1
Bacterial Stains and Plasmids

| Strain or plasmid | Relevant characteristics | Source or Reference |
|---|---|---|
| E. coli | | |
| S17-1 | recA pro hsdR RP4-2-Tc::Mu-Km::Tn7; mobilizer strain | Simon et al., 1983 |
| DH5αMCR | F2 mcrA D(mrr-hsdRMS-mcrBC) f80dlacZDM15 D(lacZYA- | Bethesda Research |
| | argF)U169 deoR recA1 endA1 supE44 12 thi-1 gyrA96 relA1 | Laboratories |
| XL10 Gold Kan^{R} | Tetr Δ(mcrA)183 Δ(mcrCB-hsdSMR-mrr)173 endA1 | Stratagene, La Jolla |
| | supE44 thi-1 recA1 gyrA96 relA1 lac Hte | CA |
| | [F′ proAB lacIqZΔM15 Tn10 (Tetr) Tn5 (Kanr) Amy]. | |
| S. leopoliensis UTCC 100 | Synonym S. elongatus PCC 7942 | University of |
| | | Toronto culture |
| | | Collection |
| ::P_{lac}-acsABΔC | Transgenic strain with the acsABΔC from A. xylinum | This Application |
| | NQ5 inserted in neutral site II. acsABΔC is fused to | |
| | the lac promoter. | |
| ::P_{rbcL}-acsABCD | Transgenic strain with the acsABCD from A. xylinum | This Application |
| | NQ5 inserted in neutral site II. acsABCD is fused to | |
| | the native rbcL promoter | |
| NS::cat | S. elongatus with the chloramphenicol acetyltransferase | This Application |
| | gene incorporated into neutral site II of the chromosome | |
| ‘NS::abΔc7S | Substrain of ::P_{lac}-acsABΔC | This Application |
| A. xylinum AY201 | Derivative of Gluconacetobacter. xylinum | Laboratory stock |
| | ATCC 23769 | |
| A. xylinum NQ5 | Also known as Gluconacetobacter xylinus | Laboratory stock |
| | ATCC 53582 | |
| pUC19 | Amp^{r}; cloning vector | Norrander et al, 1983 |
| pIS311-9 | Tet^{r}; HinDIII-BamHI acsABΔC fragment | Inder Saxena, |
| | from A. xylinum NQ5 cloned in pRK311 | This Laboratory |
| pAM1573 | Amp^{r}, Cam^{r}; NSII cargo vector, mobilizable by | Susan Golden Texas |
| | conjugation, for homologous recombination | A & M University |
| | into the chromosome of S. elongatus PCC 7942 | |
| pSAB1 | Amp^{r}; HindIII-BamHI fragment from pIS311-9 | This Application |
| | cloned in pUC19 | |
| pSAB2 | Amp^{r}, Cam^{r}; PvuII fragment from pSAB1 | This Applicaiton |
| | cloned in pAM1573 | |
| pET17b | Amp^{r}; T7-based cloning vector | Novagen |
| pET17b[P_{rbcL}] | Amp^{r}, pET17b with the strong rbcL promoter replacing | This Application |
| | the from S. leopoleinsis UTCC 100 lac promoter. | |
| pACOI | Amp^{r}, pET17b[PrbcL] with acsABCD ligated at the NdeI | This Application |
| | and BamHI sites, fusing P_{rbcL} to the operon. | |
| pACOII | Amp^{r}, Cam^{r}; XhoI-XbaI acsABCD fragment from | This Application |
| | pACOI cloned in pAM1573 | |
| pDS4101 | Amp^{r}; ColK derived helper plasmid for | Finnegan and |
| | mobilization | Sherratt, 1982 |

DNA manipulations. Genomic DNA was isolated from S. leopoliensis essentially as described by Susan Golden (Golden et al., 1987), with the exception that DNA was ethanol precipitated rather than purified using glass fines. Plasmids were isolated using Qiagen miniprep kits. Restriction enzymes and T4 DNA ligase were purchased from Promega and used following the manufacturer's instructions. Agarose gels were prepared and examined as previously described (Mantiatis et al., 1982). When more delicate handling of DNA was required, visualization of bands was accomplished via agarose gels supplemented with 40 ul of 2 mg/ml crystal violet (CV) per 50 ml agarose. When using CV gels, DNA samples were run in loading buffer composed of 30% glycerol, 20 mM EDTA, and 100 ug/ml CV. This procedure allowed direct viewing of DNA eliminating the exposure of DNA to damaging uv light in order to visualize the bands. Unless otherwise noted, the transformation of chemically competent cells was performed as described previously (Chung and Miller, 1993).

Cloning the rbcL promoter region in S. leopoliensis. Primers were designed to amplify a region 360 bp upstream of the rbcL coding region encompassing the strong rbcL promoter (PrbcL). Primer sequences were based on previous work (Deng and Coleman, 1999). PrbcL-for-XbaI (forward primer) contained a 5′ XbaI restriction site and PrbcL-rev-NdeI (reverse primer) contained a 5′ NdeI restriction site. Primer sequences were as follows: Forward primer—ACCATCTAGA-GGCTGAAAGTTTCGGACT, Reverse primer—TTCCCATATGTCGTCTCTCCCTA-GAGATATG. Restriction sites are shown in bold. The PCR product was digested and ligated into corresponding restriction sites of plasmid pET17b (Novagen) to create plasmid pET17b[PrbcL].

Cloning the acsABCD operon. The cellulose synthase operon of A. xylinum (NQ5) was amplified using overlap extension PCR consisting of three steps (Shevchuk, 2004). The first step consisted of two reactions: Reaction L amplified nucleotides 1-6090 of the acsABCD operon using primers acsABLF1 and acsABLR1, Reaction R amplified nucleotides 4594-10,094 using primers acsCDRF1 and acsCDRR1. 50 ul reaction conditions: 10 ul 10× Pfx Reaction Buffer, 1.5 ul 10 mM mixed dNTP (BD Biosciences), 1.0 ul 50 mM MgSO4, 0.3 ul of each primer (50 uM), 0.25 ul of NQ-5 DNA, and 0.5 ul Platinum Pfx (Invitrogen). Reaction L contained 15 ul Enhancer solution and 21.15 ul H₂O. Reaction R contained 17.5 ul Enhancer solution and 18.65 ul H2O. Cycling conditions: Initial denaturation 95° C. 5 min, subsequent cycles 95° C. for 15 s, annealing 60° C. for 30 s, extension 68° C. for 6 min, with a final extension at 68° C. for 20 min followed by a 4° C. hold. Primer sequences were as follows: acsABLF1—TGACCAAGACAGACACGAATTCCTCTCAGGCT, acsABLF1 GTAACCATGACAGCGTCTGGCGATATGATT, acsCDRF2—TTCCTT-TCACCACCTATGCCGATCTGTC, and acsCDRR2—TCCGCCAAGCTTCAC-CAAAAACCTTTATAATTTCA. The products of L and R reactions were run on CV gels and purified using the QIAquick gel extraction kit (Qiagen). DNA was concentrated using microcon YM100 centrifugal filters (Millipore). Step 2 (Fusion A) conditions for 50 ul reactions were as follows: 18.25 ul H2O, 10 ul 10× Pfx Reaction Buffer, 1.0 ul 50 mM MgSO4, 1.25 ul of Reaction L (700 ng), 2.5 ul of Reaction R (650 ng), 15 ul of Enhancer solution, and 0.5 ul Platinum Pfx (Invitrogen). Cycling Conditions: Initial denaturation 94° C. 5 min, subsequent cycles 94° C. for 15 s, annealing 55° C. for 30 s, extension 68° C. for 5.5 min, with final extension at 68° C. for 20 min followed by a 4° C. hold. Step 3 (Fusion B) conditions for 50 ul reactions were as follows: 11.4 ul H2O 10 ul 10× Pfx Reaction Buffer, 1.0 ul 50 mM MgSO4, 10 ul of Fusion A reaction, 0.3 ul 50 mM acsA-VspI-For#4 (forward primer), 0.3 ul 50 mM acsD-BamHI-Rev#4 (reverse primer), 15 ul of Enhancer solution, and 0.5 ul Platinum Pfx (Invitrogen). Cycling Conditions: Initial denaturation 94° C. 5 min, subsequent cycles 94° C. for 15 s, annealing 55° C. for 30 s, extension 68° C. for 5.5 min, with final extension at 68° C. for 20 min followed by a 4° C. hold. Primer sequences were as follows: Forward primer—GCGGATTAATGCCAGAGGTTCGGT-CGTCAACGCAGTCA and Reverse primer—CGTGGATCCGCCGGACGCCATCG-CATCATCCAGCAT. Primers were designed with a VspI site on the 5′ end of the forward primer and a BamHI site on the 5′ end of the reverse primer. Restriction sites are shown in bold. The PCR product was digested and ligated into the corresponding restriction sites on pET17b[PrbcL] to create pACOI, placing the acsABCD operon under the control of the rbcL promoter. The ligation product was transformed into XL10 Gold KanR Competent E. coli Cells (Stratagene) using the manufacturer's instructions. pET17b[PrbcL] and pAM1573 were digested with XhoI and XbaI and the ˜10 kb PrbcL-acsABCD fragment and the cargo plasmid were ligated to create pACOII.

Construction of Cargo Plasmid pSAB2. A 5.2 kb BamHI-HindIII fragment from pIS311-9 containing acsABΔC was ligated into the BamHI-HindIII sites of pUC19 to create pSAB1. A 7.9 kb PvuII fragment from pSAB1 containing the lac operon promoter/operator with a lacZa-acsABΔC fusion was ligated into the unique SmaI site of pAM1573 to create pSAB2. See Table 1 for plasmid descriptions.

Conjugation. Conjugations transferring cargo plasmid pSAB2 were performed via biparental matings of S. leopoliensis with the E. coli strain, S17. Conjugations with pACOII were conducted using S17-1 carrying the helper plasmid pDS4101. Controls were performed using S17-1 without cargo plasmids. 1.5 ml of a S. leopoliensis culture with an OD750 of 0.4-0.6 was centrifuged at 8,000 rpm in a microfuge for 3 minutes. The pellet was resuspended in 200 ul BG11. Serial dilutions of the suspension were prepared to 10-1-10-5 in BG11 for studies and controls. 1 ml aliquots from overnight cultures of S17-1 (OD650 of 0.9-1.0) were harvested at 5,000 rpm in a microfuge for 2 min. The pellets were washed twice with 1 ml of LB followed by gentle resuspension in H₂O. 100 ul of S17-1 carrying cargo plasmid was added to each experimental dilution. 100 ul of S17-1 without cargo plasmid was added to each control dilution. 200 ul of each dilution was spread out on BG11 plates containing 5% LB. Plates were allowed to grow overnight without selection. The plates were underlaid with chloramphenicol as previously described (Golden, 1987). Putative exconjugate colonies were restreaked on BG11 with chloramphenicol selection in order to obtain S. leopoliensis colonies free from E. coli. Cultures were then examined for E. coli contamination by growth on LB plates at 37° C.

Screening for acsAB. Colonies of S. leopoliensis were prepared for PCR screens for the presence acsAB as previously described (microbiology.ucdavis.edu/meekslab/xpro6.htm). Samples were prepared in 100 ul volumes in 200 ul PCR tubes. A 1084 bp fragment spanning the acsAB genes was amplified using the primers Forward—TGGCGTGGTGTCTATGAA-CTGTCTTT and Reverse—CGGATATACTGCTCGTTCAGCGTCAT. PCR was performed using Herculase Hotstart DNA polymerase (Stratagene): 1× Herculase reaction buffer (Stratagene), 200 uM each dNTP, 0.25 uM of each primer, 2.5 U 50 ul-1 Herculase Hotstart polymerase (Stratagene), and 4% DMSO. Templates were added to 5 ul reactions as follows: 1 ul of prepared colony solution, and 0.25 ul of NQ5 genomic or plasmid DNA (˜10 ng). Reaction conditions were set up according to the manufacturer's instructions for high GC targets.

Membrane Preparations. 1 L of S. leopoliensis liquid culture (OD750 of 0.4-0.6) was harvested at 3470×g and resuspended in 5 ml 20 mM K2PO4, pH 7.8 with 3% PMSF. Crude membranes were prepared as previously described (Norling, 1998). 200 ml cultures of A. xylinum (AY201) containing 0.25% Celluclast were grown for 2 days at 28° C. Cells were collected by centrifugation at 3470×g for 10 min at 4° C., resuspended in 2 ml TME, and frozen at −80° C. Frozen cells were resuspended to 20 ml in TE and passed four times through a prechilled French pressure cell at 1200 psi. 20 ul of 3% PMSF was immediately added to the lysate. Lysate was centrifuged at 3,310×g for 10 minutes to remove cell debris. The supernatant was centrifuged at 103,000×g for 30 minutes at 4° C. Pelleted crude membranes were resuspended in 200 ul TME and frozen at −80° C. Protein concentrations of membrane fractions were determined using the BioRad DC kit following the manufacturer's instructions.

Western Analysis. Polyacrylamide gel electrophoresis was conducted as previously described (Laemmli, 1970). For Western blots, protein samples were transferred from the gels to nitrocellulose (Invitrogen) overnight at a constant current of 150 mA using a Bio-Rad Semi-Dry Transfer Cell. Western blots were performed using enhanced chemiluminescence (ECL) detection (Amersham, manufacturer's protocol). Anti −93 serum (Chen and Brown, 1996) was used a 1:30,000 dilution. The goat-anti-rabbit was used at 1:10,000 dilution.

Microscopy. Wild-type and mutant cells were collected in aliquots from liquid culture or as aqueous suspensions from plates. For fluorescence microscopy, cells were labeled with 100 uM Tinopal LPW and viewed at 365 nm excitation wavelength. For TEM preparations, CBHI-gold labeling was performed essentially as described previously (Okuda et al., 1993) with the following exceptions: (1) 10 nm gold was used for the CBHI-gold complex, (2) rather than floating grids, 6 ul drops of enzyme complex were added to Formvar grids, and (3) enzyme complex and product were incubated for 1 min at room temperature. Grids were negative stained with 2% uranyl acetate.

### EXAMPLE 2

Genetically modified strains of Synechococcus (see Table 1 for a description of strains) were maintained at 2⁴° C. with 12 hour light/dark cycles using BG11 (Allen, 1968) as the growth medium. Solid media was prepared with 1.5% agar as previously described (Golden, 1988). 50 ml liquid cultures were maintained on a rotary shaker in 250 ml Erlenmeyer flasks. Growth media was supplemented with 7.5 ug/ml chloramphenicol. Cell concentrations of cultures were determined by measuring their optical density at 750 nm (OD₇₅₀).

Celluclast Digestions. Celluclast (Sigma C2730) was diluted 1:1 in 20 mM Sodium Acetate, pH 5.2 and sterilized by passage through a 0.2 um filter (Pall Life Sciences PN 4433). 50 ml cultures of NS::cat and NS::abΔc7S were grown to stationary phase under the conditions described above. The OD750 of each culture was recorded. 40 ml of each culture was centrifuged (10 min, RT, 1,744×g) in and IEC clinical centrifuge. The supernatants were discarded, wet weights recorded, and the pellets resuspended in 10 mM Sodium Acetate, pH 5.2. For buffer-only samples, 250 ul aliquots were transferred to 1.5 ml Eppendorf tubes. For Celluclast digestions, 247.5 ul of resuspended cells and 2.5 ul of sterilized Celluclast were combined in 1.5 ul eppendorf tubes. Enzyme blanks containing only Celluclast and buffer were also prepared. The tubes were placed on a rotisserie and incubated overnight at 30° C. under constant illumination

Glucose Assays. After overnight incubation, cells were pelleted by centrifugation (5 min, RT, 14,000 rpm) in a microcentrifuge. The supernatant was carefully pipetted off the cell pellet and retained for the glucose assay. Glucose concentration was measured using the hexokinase-glucose 6-phosphate dehydrogenase enzymatic assay (Sigma G3293). Assays were performed with 50-100 ul of supernatant per reaction following the manufacturer's instructions. Final glucose concentrations were determined by subtracting the glucose content of the Celluclast enzyme blank from the gross cyanobacterial glucose concentrations.

Upon lossless scale-up, the preliminary results presented in Table 2 suggest a yield of approximately 85 gallons of ethanol acre foot-1 year-1. This is significantly less than predicted yields for switchgrass (1150 gallons acre-1 year-1). However Synechococcus (strain NS::abΔc7S) possesses several advantageous characteristics which may allow it to be competitive with land-based crops: (1) It possesses a rapid generation time; (2) It can be grown in brackish water; (3) the cellulose synthesized by this organism can be hydrolyzed by cellulytic enzymes without the pretreatment procedures required when utilizing lignocellulosic feedstocks, such as switchgrass, for ethanol production; and (4) after digestion with cellulases, cells can be returned unharmed to photobioreactors for continued cellulose production. Additionally, this organism is amenable to genetic manipulation by both natural transformation and conjugation. Thus, the potential for increased production by genetic manipulation exists.

TABLE 2
Amount of glucose liberated from extracellular polysaccharides (EPS)
by Celluclast digestion. Glucose from EPS was determined by subtracting the
concentration of glucose present in the buffer-only sample from the total glucose
measured in the Celluclast digestions.

| | | Wet | Glucose mg/ml - | Total Glucose mg/ml - | Glucose |
|---|---|---|---|---|---|
| | | Weight | Sodium Acetate- | Celluclast | mg/ml from |
| | OD₇₅₀ | (g) | only | digestion | EPS |
| NS::cat | 1.00 +/− 0.18 | 0.19 +/− 0.08 | 0.03 +/− 0.04 | 0.08 +/− 0.03 | 0.05 +/− 0.03 |
| NS::abΔc7S | 1.20 +/− 0.19 | 0.20 +/− 0.07 | 0.09 +/− 0.06 | 0.31 +/− 0.012 | 0.22 +/− 0.06 |

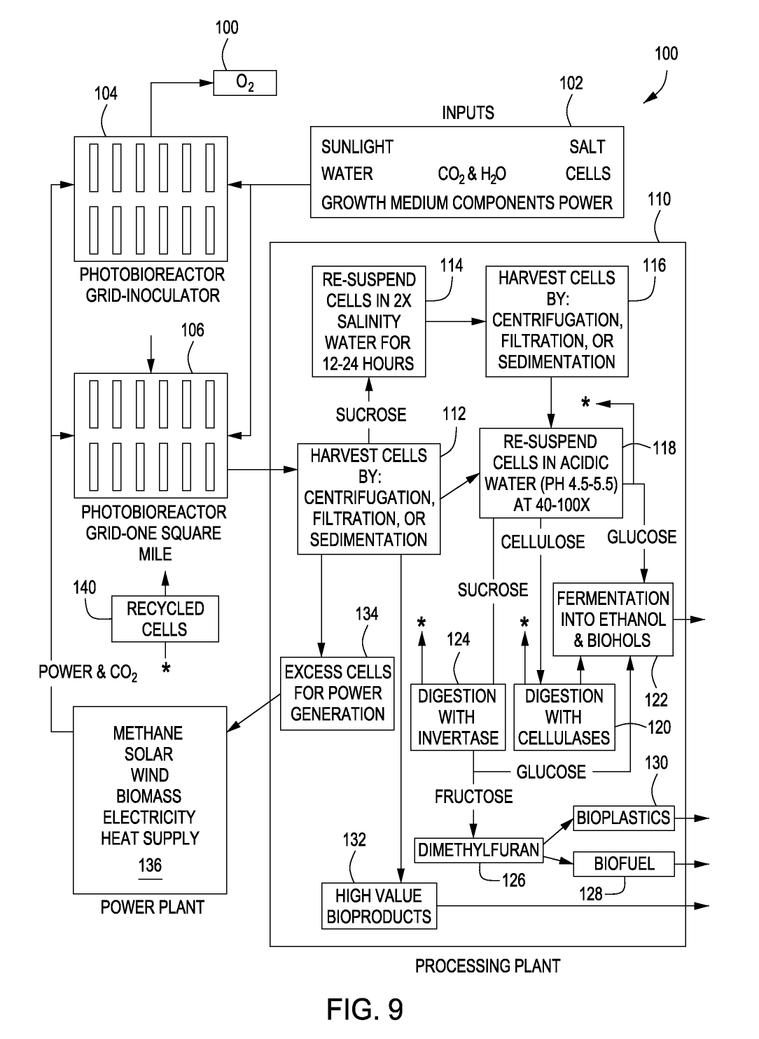

FIG. 9 shows one example of a photobioreactor system 100 of the present invention. First, inputs 102 for the photobioreactor system may include: sunlight, salt, water, CO₂ modified-cyanobacterial cells of the present invention, growth medium components and if necessary a source of power to move the components (e.g., pumps or gravity). Next, the inputs 102 and inoculated into a photobioreactor grid 104 that is used to grow the modified-cyanobacteria in size and number, to test for saccharide production and to reach a sufficiently high enough concentration to inoculate the operating photobioreactor 106. The photobioreactor 106 may be a pool or pool(s), trench or other vessel, indoor or outdoor that is used to grow and harvest a sufficient volume of photobiomass for subsequent processing in, e.g., processing plant 110. In one example, the photobioreactor 106 may be a grid of pools of one square mile (or larger) that may be used in parallel or in series to produce the photobiomass. Depending on the geographical location of the photobioreactor 106, the water may be saltwater or brine obtained from a sea that is gravity fed into the pools. Gravity or pumping may be used, however, gravity has the advantage that it does not require additional energy to move the photobiomass from pool to pool and even into the processing plant. In fact, in certain embodiments the entire system may be gravity fed with the final products gravity fed into underground rivers that return to the sea or ocean.

The processing plant 110 includes a cell harvested 112, which may allows the isolation of the photobiomass by, e.g., centrifugation, filtration, sedimentation, creaming or any other method for separating the photobiomass, the modified-cyanobacterial cells and the liquid. For the isolation of sucrose, the cells may be resuspended in medium with an increased salinity 114 (e.g., 2× the salinity) followed by a second harvesting step 116. The twice-harvested cells are then resuspended under acidic conditions (e.g., pH 4.5-5.5) at 40 to 100× the concentration and the sucrose is secreted by the modified-cyanobacteria. If glucose is preferred, the once harvested cells are resuspended under acidic conditions 118 and glucose is secreted. In addition, whether sucrose or glucose is secreted, cellulose is also harvested from the modified-cyanobacteria, which may be further digested by cellulases 120. Glucose and digested cellulose can then be fermented into ethanol or other alkanols.

If sucrose is secreted and obtained, then the sucrose can be converted into dimethylfuran and glucose by invertase 124. The methylfuran 12 can then be used for bioplastic 130 or biofuel 128 production. Glucose that is obtained after the invertase reaction 124 can then be directed back into the fermentation reactions.

In addition to the production of ethanol, bioplastics and other biofuels, the harvested cells can he used for the production of other high value bioproducts, e.g., by further modifying the microbial cellulose-producing cyanobacteria to make other bioproducts, e.g., pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites. After each of these steps, the modified-cyanobacteria can then be recycled into the photobioreactors for additional carbon fixation. Furthermore, the products of the processing plant 110 can also be combined with other power sources, e.g., solar, methane, wind, etc., to generate electricity and heat (in addition to recycling any CO₂ released in the processing plant 110), to power the inoculation pool 104 and the photobioreactor 106.

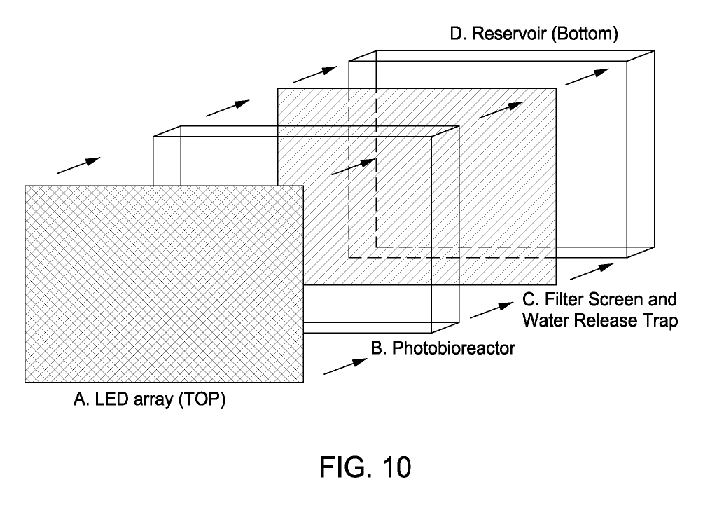

FIG. 10 shows a photobioreactor design for the in situ harvest of cyanobacterial saccharides. The photobioreactor complex can be located indoors or underground. Part A is an LED array, powered by photovoltaic cells, provides mono or polychromatic light at a pulsed frequencies corresponding to the rate limiting steps of photosynthesis for maximized photosynthetic productivity. Part B is a transparent photobioreactor acting as a growth vessel for cyanobacterial cells. The horizontal orientation of the photobioreactor allows for efficient separation of cells from culture medium by use of gravity and air pressure. Part C is a filter screen combined with a water release trap that will separate cells from the culture medium. The filter screen will have pore sizes capable of retaining cyanobacterial cells while allowing culture medium to flow into the reservoir. The transfer will be facilitated by gravity and air pressure generated by closing the gas outlet of the photobioreactor. The reservoir, located beneath the photobioreactor, will act to retain culture medium during harvest of saccharides. After harvest, culture medium will be returned to the photobioreactor from the reservoir via pump.

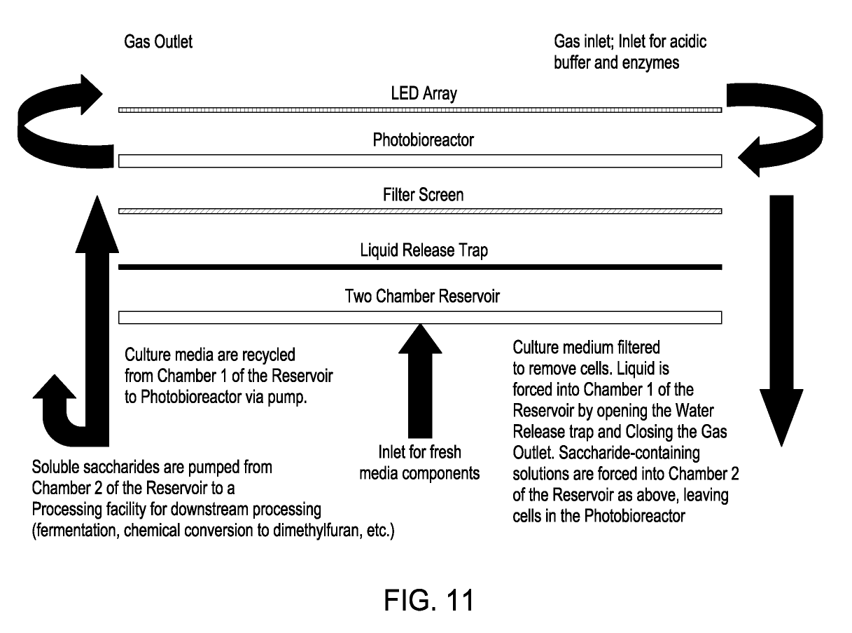

FIG. 11 shows the operation of a photobioreactor complex design for in situ harvest of cyanobacterial saccharides. The LED array, located on top of the photobioreactor complex will supply pulsed mono or polychromatic light for maximum photosynthetic conversion efficiency. Air flow (CO₂, N₂, or ambient air) delivered by the gas inlet during growth periods will serve to deliver carbon and/or nitrogen sources for fixation and created turbulence for maintaining cell suspension. A gas outlet will facilitate the release of waste gasses (O₂ and H₂) that are potentially detrimental to the cyanobacterial growth and relieve excess air pressure from the system during growth phases. Removal of culture media for harvesting of saccharides will be facilitated by the opening of the liquid release trap coupled with closing the gas outlet. The increase in air pressure, combined with gravity, will force the culture medium through the filter which will retain cyanobacterial cells. Cyanobacterial cells can then be resuspended in specific buffer or media designed for cellulose digestion or the direct secretion of saccharides. The saccharide-containing solutions will be drained to chamber 2 of the liquid release trap by the same method described for growth media above. Soluble saccharides will be pumped from chamber 2 of the reservoir to central processing units for downstream conversion processes (e.g., fermentation, chemical conversion to dimethylfuran, etc.). Cells will be resuspended by closing the water release trap and pumping culture medium which has been recombined with fresh media components (e.g., nitrates, phosphates, etc.) from chamber 1 of the reservoir.

Another embodiment of the present invention includes a method of fixing carbon by growing a sucrose-producing cyanobacterium in a CO₂-containing growth medium; generating sucrose with said cyanobacterium, wherein CO₂ is fixed into sucrose at a level higher than an unmodified cyanobacterium; and calculating the amount of CO₂ fixed into the sucrose to equate to one or more carbon credit units. For example, at least one other carbon may be fixed into sucrose and the at least one other carbon's is equated to carbon credit units that is included in the calculation. The method may further include the step of processing the sucrose into ethanol, e.g., as a renewable feedstock for biofuel production. Generally, the cyanobacterium fixes CO₂ and thus atmospheric CO₂ using the present invention into a renewable feedstock of saccharides for, e.g., animals. Importantly, it has been found that the cyanobacteria of the present invention produce sucrose, but also secrete the sucrose into the medium under certain conditions.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

Andersson C A, Tsinoremas N F, Shelton J, Lebedeva N V, Yarrow J, Min H, Golden S S. (2000). Application of bioluminescence to the study of circadian rhythms in cyanobacteria. In Zielger M M and Goldwin T O (eds) Methods in Enzymology Vol. 305 pp 527-42. Academic Press, Inc. New York.
Asada T, Koike Y, Schnackenberg J, Miyake M, Uemura I, Miyake J. (2000). Heterologous expression of clostridial hydrogenase in the cyanobacterium Synechococcus PCC 7942. Biochim Biophys Acta. 1490: 269-278.
Bajpai P. (2004). Biological bleaching of chemical pulps. Crit. Rev Biotechnol. 24(1):1-58.
Brown Jr R M. (2004). Position paper: microbial cellulose a new resource for wood, paper, textiles, food and specialty products visit: <www.botany.utexas.edu/facstaff/facpages/mbrown/position1.htm>.
Brown Jr R M and Lin F C. (1990). Multiribbon microbial cellulose. U.S. Pat. No. 4,954,439.
Brown Jr R M, Willison J H M, C L Richardson. (1976). Cellulose biosynthesis in Acetobacter xylinum: 1. Visualization of the site of synthesis and direct measurement of the in vivo process. Proc Nat Acad Sci USA. 73(12): 4565-4569.
Castenholz R W, Waterbury J B. (1989). Group I. Cyanobacteria. In: Staley J T, Bryant M P, Pfennig N, and Holt J G (eds) Bergey's Manual of Systematic Bacteriology, Vol 3, pp 1710-1728. Williams and Wilkins Co, Baltimore.
Chen H P, Brown Jr R M. (1996). Immunochemical studies of the cellulose synthase complex in Acetobacter xylinum. Cellulose. 3:63-76.
Chung C T, Miller R H. (1993). Preparation and storage of competent Escherichia coli cells. In: Wu R (ed) Methods in Enzymology Vol. 218 pp 621-627 Academic Press, Inc. New York.
Cogne G, Comet J F, Gros J B. (2005). Design, operation, and modeling of a membrane photobioreactor to study the growth of the Cyanobacterium Arthrospira platensis in space conditions. Biotechnol Prog. 21(3): 741-50.
Czaja W, Krystynowicz A, Bielecki S, Brown Jr R M. (2006). Microbial cellulose—the natural power to heal wounds. Biomaterials. 27: 145-151.
Deng M D, Coleman J R. (1999). Ethanol synthesis by genetic engineering in cyanobacteria. Appl Environ Microbiol. 65(2): 523-8.
Eriksson I S, Elmquist H, Nybrant T. (2005). SALSA: a simulation tool to assess ecological sustainability of agricultural production. Ambio. 34(4-5): 388-92.
Galperin M Y, Nikolskaya A N, Koonin E V. (2001). Novel domains of the prokaryotic two-component signal transduction systems. FEMS Microbiol Lett. 203(1): 11-21.
Golden S S, Brusslan J, Haselkom R. (1987). Genetic engineering of the cyanobacterial chromosome. In Wu R and Grossman L (eds) Methods in Enzymology Vol. 153 pp 215-231. Academic Press, Inc. New York
Golden S S. (1988). Mutagenesis of cyanobacteria by classical and gene-transfer-based methods. In Packer L and Glazer A N (eds) Methods in Enzymology ed. Vol. 167 pp 714-727. Academic Press, Inc. New York.
Golden S S, Johnson C H, Kondo T. (1998). The cyanobacterial circadian system: a clock apart. Curr Opin Microbiol. 1: 669-73.
Helenius G, Bäckdahl H, Bodin A, Nannmark U, Gatenholm P, Risberg B. (2006). In vivo biocompatibility of bacterial cellulose. Biomed Mater Res A. 76(2): 431-8.
Hess K, Haller R, Katz J R. (1928). Die Chemie der Zelluloseund ihrer Begleiter. Akademische Verlagsgesellschaft, Leipzig.
Kim S G, Choi A, Ahn C Y, Park C S, Park Y H, Oh H M. (2005). Harvesting of Spirulina platensis by cellular flotation and growth stage determination. Lett Appl Microbiol. 40(3): 190-4.
Klemm D, Heublein B, Fink H P, Bohn A. (2005). Cellulose: Fascinating Biopolymer and Sustainable Raw Material. Angew Chem. Int. 44: 3358-3393.
Kondo T, Togawa E, Brown Jr R M. (2001). “Nematic ordered cellulose”: a concept of glucan chain association. Biomacromolecules. 2(4): 1324-30.
Kuhlemeier C J, van Arkel G A. (1987). Host-vector systems for gene cloning in cyanobacteria. Methods Enzymol. In: Wu R and Grossman L (eds) Methods in Enzymology Vol. 153 pp 199-215. Academic Press, Inc. New York.
Laemmli U K. (1970). Cleavage of structural proteins during the assembly of the head of bacteriophage T4. Nature. 227: 680-685.
Li R, Golden S S. (1993). Enhancer activity of light-responsive regulatory elements in untranslated leader regions of cyanobacterial psbA genes. Proc Natl Acad Sci USA. 90:11678-11682.
Lin F C, Brown Jr. R M. (1989). Purification of cellulose synthase from Acetobacter xylinum. In: Schuerch C (ed). Cellulose and Wood-Chemistry and Technology. pp 473-492. John Wiley and Sons, Inc. N.Y.
Lynd L R, Weimer P J, van Zyl W H, Pretorius I S. (2002). Microbial cellulose utilization: fundamentals and biotechnology. Microbiol. Mol Biol Rev. 66(3): 506-577.
Mantiatis T, Fritsch E, Sambrook J. (1982). Molecular Cloning (A laboratory manual). Cold Spring Harbor Laboratory.
Mermet-Bouvier P, Chauvat F. (1994). A conditional expression vector for the cyanobacteria Synechocystis sp. Strains PCC6803 and PCC6714 or Synechococcus sp. Strains PCC7942 and PCC6301. Curr Microbiol. 28: 145-148.
Moreno J, Vargas M A, Rodriguez H, Rivas J, Guerrero M G. (2003). Outdoor cultivation of a nitrogen-fixing marine cyanobacterium, Anabaena sp. ATCC 33047. Biomol Eng. 20(4-6): 191-7.
Murphy R C, Stevens Jr S E. (1992) Cloning and expression of the cryIVD gene of Bacillus thuringiensis subsp. israelensis in the cyanobacterium Agmenellum quadruplicatum PR-6 and its resulting larvicidal activity. Appl Environ Microbiol. 58(5): 1650-5.
Nair U, Thomas C, Golden S S. (2000). Functional elements of the strong psbAI promoter of Synechococcus elongatus PCC 7942. J. Bacteriol. 83(5): 1740-7.
Norander J, Kempe T, Messing J. (1983). Construction of improved M13 vectors using oligodeoxynucleotide-directed mutagenesis Gene. 26: 101-106.
Norling B, Zak E, Andersson B, Pakrasi H. (1998). 2D-isolation of pure plasma and thylakoid membranes from the cyanobacterium Synechocystis sp. PCC 6803. FEBS Lett. 436: 189-192.
Peng L, Kawagoe Y, Hogan P, Delmer D. (2002). Sitosterol-beta-glucoside as primer for cellulose synthesis in plants. Science. 295(5552): 147-50.
Peng L, Xiang F, Roberts E, Kawagoe Y, Greve L C, Kreuz K, Delmer D P. (2001). The experimental herbicide CGA 325'615 inhibits synthesis of crystalline cellulose and causes accumulation of non-crystalline beta-1,4-glucan associated with CesA protein. Plant Physiol. 126(3): 981-92.
Römling U, Gomelsky M, Galperin M. (2005). C-di-GMP: the dawning of a novel bacterial signaling system. Mol. Microbiol. 57(3): 629-639.
Ross, P, Weinhouse H, Aloni Y, Michaeli D, Weinberger-Ohana P, Mayer, Braun R S, de Vroom E, van der Marel G A, van Boom J H, Benziman M. (1987). Regulation of cellulose synthesis in Acetobacter xylinum by cyclic diguanylic acid. Nature. 325: 279-281.
Saxena I M, Kudlicka K, Okuda K, Brown Jr R M. (1994) Characterization of genes in the cellulose synthesizing operon (acs operon) of Acetobacter xylinum: implications for cellulose crystallization. J. Bacteriol. 176: 5735-5752.
Shah J, Brown Jr R M. (2005). Towards electronic paper displays made form microbial cellulose. Appl Microbiol Biotechnol. 66: 352-355.
Shestakov S V, Khyen N T. (1970). Evidence for genetic transformation in blue-green alga Anacystis nidulans R2. Mol Gen Genet. 107: 372-5.
Shevchuk N A, Bryksin A V, Nusinovich Y A, Cabello F C, Sutherland M, Ladisch S. (2004). Construction of long DNA molecules using long PCR-based fusion of several fragments simultaneously. Nuc Acids Res. 32(2) e19.
Shramm M, Hestrin S (1954). Factors effecting production of cellulose at the air/liquid interface of a culture of Acetobacter xylinum. J Gen Microbiol. 11: 123-129.
Simon R, Priefer U, Pühler A. (1983). A broad host range mobilization system for in vivo genetic engineering: transposon mutagenesis in gram negative bacteria. BioTechnology. 1: 784-791.
Sippola K, Kanervo I, Murata N, Aro E. (1998). A genetically engineered increase in fatty acid unsaturation in Synechococcus sp. PCC 7942 allows exchange of D1 protein forms the sustenance of photosystem II activity at low temperature. Eur J Biochem. 251: 641-648.
Tabuchi M, Kobayashi K, Fugimoto M, Baba Y. (2005). Bio-sensing on a chip with compact discs and nanofibers. Lab Chip. 5(12): 1412-1415.
Tal R, Wong H C, Calhoon R, Gelfand D, Fear A L, Volman G, Mayer R, Ross P, Amikam D, Weinhouse H, Cohen A, Sapir S, Ohana P, Benziman M. (1998) Three cdg operons control cellular turnover of cyclic di-GMP in Acetobacter xylinum: genetic organization and occurrence of conserved domains in isoenzymes. J Bacteriol. 180(17): 4416-4425.
Thiel T. (1994). Genetic analysis of Cyanobacteria. In: Bryant D A (ed) The Molecular Biology of Cyanobacteria. pp 582-606. Kluwer Academic Publishers, Boston.
Vincent W F. (2000). Cyanobacterial dominance in the polar regions. In: Whitton B A, Potts M (eds) The Ecology of Cyanobacteria. pp 321-340. Kluwer Academic, The Netherlands.

Weinhouse H, Sapir S, Amikam D, Shilo Y, Volman G, Ohana P, Benziman M. (1997). c-di-GMP-binding protein, a new factor regulating cellulose synthesis in Acetobacter xylinum. FEBS Lett. 416: 207-211.
White D G, Brown Jr R M. (1989). Prospects for the commercialization of the biosynthesis of microbial cellulose. In: Schuerch C (ed). Cellulose and Wood-Chemistry and Technology. 573-590. John Wiley and Sons, Inc. N.Y.
Wynn-Williams D D. (2000). Cyanobacteria in Deserts—Life at the Limit? In: Whitton B A, Potts M (eds) The Ecology of Cyanobacteria. pp 341-361. Kluwer Academic, The Netherlands.
Yamada Y, Hoshino K, Ishikawa T. (1997). The phylogeny of acetic acid bacteria based on the partial sequences of 16S ribosomal RNA: the elevation of the subgenus Gluconacetobacter to the generic level. Biosci Biotechnol Biochem. 61(8): 1244-51.
Zaar K. (1979). Visualization of pores (export sites) correlated with cellulose production in the envelope of the gram-negative bacterium Acetobacter xylinum. J Cell Biol. 80(3): 773-7.
Zogaj X, Nimtz M, Rohde M, Bokranz W, Römling U. (2001). The multicellular morphotypes of Salmonella typhimurium and Escherichia coli produce cellulose as the second component of the extracellular matrix. Mol. Microbiol. 39: 1452-63.

What is claimed is:

1. A cyanobacterium comprising a portion of an exogenous cellulose operon sufficient to express bacterial cellulose.

2. The cyanobacterium of claim 1, wherein the cyanobacteria comprises a photosynthetic cyanobacterium, a nitrogen-fixing cyanobacterium, a cyanobacterium capable of growing in brine, a cyanobacterium that is a facultative heterotroph, a cyanobacterium that is chemoautotrophic, and combinations thereof.

3. The cyanobacterium of claim 1, wherein the cyanobacteria comprise a photosynthetic cyanobacterium Synechococcus sp.

4. The cyanobacterium of claim 1, wherein the portion of the cellulose operon sufficient to express bacterial cellulose comprises the acsAB genes from the cellulose synthase operon stably integrated into the chromosome.

5. The cyanobacterium of claim 1, wherein the cellulose operon comprises P_{lac}-acsABΔC.

6. The cyanobacterium of claim 1, wherein the cellulose operon comprises an acsABCD operon under control of an PrbcL promoter from Synechococcus leopoliensis.

7. The cyanobacterium of claim 1, wherein the cellulose operon comprises an acsABCD operon from Acetobacter strain NQ5.

8. The cyanobacterium of claim 1, wherein the cellulose operon comprises an acsABCD from NQ5 under the control of a PrbcL promoter from Synechococcus leopoliensis.

9. The cyanobacterium of claim 1, wherein the portion of the cellulose operon sufficient to express bacterial cellulose comprises the acsAB genes from the cellulose synthase operon of Acetobacter sp.

10. The cyanobacterium of claim 1, wherein the portion of the cellulose operon sufficient to express bacterial cellulose comprises the acsAB genes from the cellulose synthase operon of the gram negative bacterium Acetobacter xylinum.

11. The cyanobacterium of claim 1, wherein the cellulose comprises crystalline native cellulose I, regenerated and native cellulose II, nematic ordered cellulose, a glucan chain association, cellulose acetate and combinations thereof.

12. The cyanobacterium of claim 1, wherein the cellulose synthesizing enzymes are from mosses (Physcomitriella), algae, ferns, vascular plants, tunicates, gymnosperms, angiosperms, cotton, switchgrass and combinations thereof.

13. A method of producing cellulose comprising:
expressing in a photosynthetic cyanobacterium a portion of the cellulose synthesizing enzymes or operon sufficient to express bacterial cellulose; and isolating the cellulose produced by the photosynthetic cyanobacterium.

14. The method of claim 13, wherein the cyanobacteria comprises a photosynthetic cyanobacterium Synechococcus sp.

15. The method of claim 13, wherein the portion of the cellulose operon sufficient to express bacterial cellulose comprises the acsAB genes from the cellulose synthase operon stably integrated into the chromosome.

16. The method of claim 13, wherein the cellulose operon comprises P_{lac}-acsABΔC.

17. The method of claim 13, wherein the portion of the cellulose operon sufficient to express bacterial cellulose comprises the acsAB genes from the cellulose synthase operon of Acetobacter sp.

18. The method of claim 13, wherein the cellulose has a lower crystallinity than wild-type bacterial cellulose and the lower crystallinity cellulose is degraded with less energy into glucose than wild-type cellulose.

19. The method of claim 13, wherein the cellulose has a lower crystallinity than wild-type bacterial cellulose and the lower crystallinity cellulose is degraded with less energy into glucose than wild-type cellulose and is further converted into ethanol to be used as a biofuel and, optionally, that the cells are returned unharmed to the growth medium for continued cellulose and biomass production.

20. A Synechococcus sp. cyanobacterium comprising one or more genes from the acsAB cellulose synthase operon from a bacterium under the control of a promoter such that the cyanobacteria expresses bacterial cellulose.

21. A system for the manufacture of bacterial cellulose comprising:
growing an exogenous cellulose expressing cyanobacterium in ponds or enclosed photobioreactors exposed to natural sunlight or artificial light generated by LEDs or other devices; and harvesting from the ponds and/or enclosed photobioreactors the cyanobacteria and their exogenous cellulose and/or value added products.

22. The system of claim 21, wherein the exogenous cellulose expressing cyanobacterium is adapted for growth in a hypersaline environment, such that the cyanobacterium does not grow in a fresh water or a sea water salinity.

23. The system of claim 21, wherein the exogenous cellulose expressing cyanobacterium is auxotrophic for an amino acid, nucleic acid, a source of nitrogen, a source of sulfur, a mineral, a vitamin or a metal.

24. The system of claim 21, wherein the exogenous cellulose expressing cyanobacterium sequesters CO₂ thereby reducing greenhouse gasses responsible for global warming.

25. The system of claim 21, wherein the exogenous cellulose expressing cyanobacterium is grown in anywhere in the world as a novel large scale source of cellulose for wood, cotton replacements, biofuels, or value added products including but not limited to: pharmaceuticals and/or vaccines, vitamins, industrial chemicals, proteins, pigments, fatty acids and their derivatives (such as polyhydroxybutyrate), acylglycerols (as precursors for biodiesel), as well as other secondary metabolites.


SEQUENCE LISTING (10 sequences)

SEQ ID NO: 1 - DNA, 28 bp, Artificial
  synthetic oligonucleotide
accatctagaggctgaaagtttcggact

SEQ ID NO: 2 - DNA, 31 bp, Artificial
  synthetic oligonucleotide
ttcccatatgtcgtctctccctagagatatg

SEQ ID NO: 3 - DNA, 32 bp, artificial
  synthetic oligonucleotide
tgaccaagacagacacgaattcctctcaggct

SEQ ID NO: 4 - DNA, 30 bp, artificial
  synthetic oligonucleotide
gtaaccatgacagcgtctggcgatatgatt

SEQ ID NO: 5 - DNA, 28 bp, artificial
  synthetic oligonucleotide
ttcctttcaccacctatgccgatctgtc

SEQ ID NO: 6 - DNA, 35 bp, artificial
  synthetic oligonucleotide
tccgccaagcttcaccaaaaacctttataatttca

SEQ ID NO: 7 - DNA, 38 bp, artificial
  synthetic oligonucleotide
gcggattaatgccagaggttcggtcgtcaacgcagtca

SEQ ID NO: 8 - DNA, 36 bp, artificial
  synthetic oligonucleotide
cgtggatccgccggacgccatcgcatcatccagcat

SEQ ID NO: 9 - DNA, 26 bp, artificial
  synthetic oligonucleotide
tggcgtggtgtctatgaactgtcttt

SEQ ID NO: 10 - DNA, 26 bp, artificial
  synthetic oligonucleotide
cggatatactgctcgttcagcgtcat